(19) United States
(12) Patent Application Publication
(10) Pub. No.: US 2015/0272485 A1
(43) Pub. Date: Oct. 1, 2015

(54) SYSTEM AND METHODS FOR AUTOMATED HEARING SCREENING TESTS

(71) Applicant: Mr. Michael Shlomo Navat, Tel-Aviv Yafo (IL)

(72) Inventors: Michael Shlomo NAVAT, Tel-Aviv Yafo (IL); Yael Levit, Tel-Aviv Yafo (IL)

(73) Assignee: Michael Shlomo Navat

(21) Appl. No.: 14/669,180

(22) Filed: Mar. 26, 2015

Related U.S. Application Data
(60) Provisional application No. 61/972,242, filed on Mar. 29, 2014.

(51) Int. Cl. A61B 5/12; A61B 5/00

(52) U.S. Cl. CPC A61B 5/123; A61B 5/742; A61B 5/7405; A61B 5/7475; A61B 2560/0242

(57) ABSTRACT

A computer-implemented method and a computerized system for performing a hearing test of a patient. The method comprises repetitively performing a plurality of iterations, each iteration including: displaying a plurality of images and consequently audibly introducing to the patient by sounding in a fixed predetermined audio intensity at least one word corresponding to at least one image of the plurality of images; responsive to the sounding the at least one word, acquiring from the patient at least one input, said input indicative of the at least one image; and determining a result of the hearing test based on a number of correct inputs provided by the patient.

### CROSS-REFERENCE TO RELATED APPLICATIONS

This application claims the benefit of priority from U.S. Provisional Application No. 61/972,242, filed Mar. 29, 2014.

### FIELD OF THE INVENTION

The present disclosure relates to auditory tests, and more particularly, to automatic systems for hearing screening tests and methods for performing automatic hearing screening tests.

### BACKGROUND OF THE INVENTION

Programs for early hearing detection and intervention are implemented in many states all over the world. The goal of early hearing detection and intervention is to maximize linguistic competence and literacy development for children who are deaf or hard of hearing. Without proper hearing rehabilitation, these children may fail to fulfill their intellectual and social potential and may have poorer educational and employment opportunities as adults.

A fundamental part of all early hearing detection and intervention programs is hearing screening, which includes tests that indicate if a person may have hearing loss. According to recommendations of the American Academy of Pediatrics (AAP), all children, with or without risk indicators, should be monitored for hearing loss, developmental milestones and hearing skills during routine medical care. This Approach that all children should be monitored is aimed to permit the detection of children with either missed neonatal hearing loss or a delayed-onset hearing loss.

Newborn hearing screening tests include, for example, otoacoustic emissions (OAE) and auditory brainstem response (ABR). The otoacoustic emissions test is often part of a newborn hearing screening program because it may detect blockage in the outer ear canal, as well as the presence of middle ear fluid and damage to the outer hair cells in the inner ear (cochlea). An earphone and microphone are placed in the ear, sounds are played and a response is measured. When the hearing is normal, an echo is reflected back into the ear canal and may be measured by the microphone but no echo is reflected when the patient suffers of hearing loss. In addition to receiving sound, a normal cochlea also emits low-intensity sounds called otoacoustic emissions. People with normal hearing produce these low-intensity emissions, but those with a hearing loss greater than 25-30 dB, do not produce any emissions.

The auditory brainstem response test is performed by pasting electrodes on the head and recording brain wave activity in response to sound. The patient rests quietly or sleeps while the test is performed.

Adult hearing screening tests include, for example, audiometry tests, which determine a patient's hearing levels with an audiometer, but may also measure ability to discriminate between different sound intensities, recognize pitch, or distinguish speech from background noise.

Behavioral hearing tests require the patient to reliably demonstrate a change in behavior when a test sound is heard. One exemplary diagnostic behavioral hearing test consists of a pure tone audiometry test and a speech audiometry test. In the pure tone audiometry test, a patient needs to indicate when hearing the stimulus, e.g. by pushing a button or raising a hand. The lowest intensity of sound heard by a patient in at least 2 out of 3 presentations is considered to be the hearing threshold. The patient's hearing thresholds for pure tone stimuli at octave frequencies between 250-8000 Hz are tested.

In the speech audiometry test, the patient needs to repeat words that are audibly presented. This test consists of two subtests: in a speech reception threshold (SRT) test, the speech reception threshold is determined by seeking the lowest audio intensity in which a patient can repeat at least 3 out of 6 two-syllable words audibly presented to him. In a speech discrimination test, a list of monosyllabic phonetically balanced words is presented at an audio intensity of 35 dB above speech reception threshold, and the percentage of words properly repeated by the patient is scored (known also as phonetically balanced score).

Despite the rising awareness to the need for hearing surveillance in young children, there is a lack of standard validated tools for hearing screening among this age group.

### SUMMARY OF THE INVENTION

The present disclosure is directed to a computer-implemented method and system for performing a hearing test of a patient. The method may include repetitively performing a plurality of iterations. Each iteration may include one or more of the following operations: 1) displaying a plurality of images and consequently audibly introducing to the patient by sounding in a fixed predetermined audio intensity at least one word corresponding to at least one image of the plurality of images; 2) responsive to the sounding the at least one word, acquiring from the patient at least one input, said input indicative of the at least one image; and 3) determining a result of the hearing test based on a number of correct inputs provided by the patient.

The method may further include terminating the hearing test if a number of successive incorrect responses exceeds a predetermined threshold, or if no input was received from the patient and detecting ambient noise in the test environment, wherein determining a result of the hearing test may be based on the detected ambient noise.

A test-set of images may be selected from an image database which may include a plurality of images from the image database. Each image may be associated with at least one word corresponding to the image. Images of the test-set may be displayed to the patient on a display unit.

The method may include repeating each iteration a certain number of times, e.g. predetermined number of times. In each iteration, at least one image may be selected, for example randomly selected, from the test-set. A carrying phrase may be audibly introduced to the patient, for example using a fixed predetermined audio intensity.

The predetermined intensity may be, for example, in the range of 20-35 DB HL or in the range of 25-30 DB HL, and may be calculated based on normal hearing of the ranges of a population to which patient belongs.

The plurality of images may be displayed while the carrying phrase and the at least one word corresponding an image are audibly introduced to the patient (for example the images may be displayed simultaneously, or substantially simultaneously with the audible introduction of the carrying phrase and/or the corresponding word). The carrying phrase may be randomly selected from a set of carrying phrases and may be concatenated to a corresponding word to generate a full sentence.

The method may include detecting whether input is acquired from the patient and determining whether the acquired input is correct or incorrect.

The computerized system for a hearing test of a patient, may include an audio sounding device and a processing unit configured to repetitively perform a plurality of iterations.

In each iteration the processing unit may be configured to display a plurality of images and consequently audibly introduce to the patient by sounding in a fixed predetermined audio intensity at least one word corresponding to at least one image of the plurality of images; responsive to the sounding the at least one word, the processing unit may be configured to acquire from the patient at least one input, said input indicative of the at least one image.

The processing unit may be configured to randomly select the image from the test-set and terminate the hearing test if a certain predetermined number of successive incorrect responses are detected, or if no response is detected in at least a predetermined number of iterations. The processing unit may be further configured to determine a result of the hearing test based on a number of correct inputs provided by the patient.

The computerized system may include a storage device for storing results of the hearing test and retrieval thereof and may include an input device for providing the input indicative of the at least one image by the patient, the input device may be selected for example from a touch screen, a keyboard, a joystick and a mouse.

### BRIEF DESCRIPTION OF THE DRAWINGS

Some non-limiting exemplary embodiments or features of the disclosed subject matter are illustrated in the following drawings.

References to previously presented elements are implied without necessarily further citing the drawing or description in which they appear.

FIG. 1 is a schematic illustration of an exemplary system for performing an automated hearing test, according to embodiments of the present disclosure;

FIG. 2 is a schematic illustration of an exemplary user interface for a hearing screening test, according to embodiments of the present disclosure

FIG. 3A is a flow chart of a method for performing automated hearing test, according to embodiments of the present disclosure;

FIG. 3B is a flow chart that schematically illustrates a method for performing automated hearing test, according to embodiments of the present disclosure; and

FIG. 4 is a graph including test results of an exemplary automated hearing test according to embodiments of the present disclosure.

### DETAILED DESCRIPTION

In the context of the present disclosure, without limiting, an image implies a digital image or digital representation of a picture. The picture depicting at least one visible or tangible object, for example, an animal, a scene or an item. The image may be displayed, for example, on a display unit or stored in a computerized storage unit.

In the context of the present disclosure, without limiting, a test-set of images is a set, collection or a plurality of images, wherein each image is associated with an at least one word corresponding to the image. In the context of the present disclosure, without limiting, a corresponding word, or a word corresponding to an image, implies one or more words which are related to, associated with, or describe an object, thing or scene depicted in the image. For example, for an image depicting an umbrella, a corresponding word may be “umbrella”. For an image of a smiling child, corresponding words may be “laughing”, “smiling” and/or “happy”. In these examples, the images which are described by corresponding words may be identified and indicated by the patient, unless the patient does not hear the words or is unwilling to cooperate.

In the context of the present disclosure, a verb phrase is a verb associated with any objects and other modifiers. For example, in the sentence “The tree is growing very slowly”, the verb phrase is “growing very slowly” and the verb is “growing”.

In the context of the present disclosure, without limiting, a sentence clause is a syntactic construction containing a subject (or a subject phrase) and a verb (or a verb phrase), forming part of a sentence or constituting a whole simple sentence. For example, “The man is talking a lot” is a full sentence in which “The man” is the subject and “talking a lot” is the verb phrase.

In the context of the present disclosure, without limiting, a full sentence is a sentence formulated such that it contains at least one sentence clause. For example, “The child is riding a bike”.

A carrying phase, in the context of the present disclosure, is a phrase that includes at least a verb phrase or a verb. Exemplary carrying phrases are: “Please point out the . . . ” (verb phrase), “where is the . . . ” (verb phrase), or “show me the . . . ” (verb). The carrying phrase may be concatenated to a subject, which is a corresponding word associated with the represented image, in order to generate a sentence.

In the context of the present disclosure, audibly presenting or audibly introducing imply sounding a noise, vocal utterance, musical tone, or the like, e.g. by an audio sounding device such as headphones, a loudspeaker or any other device which is adapted to produce sounds.

For brevity and clarity and without limiting, in the present disclosure a predetermined audio intensity is an audio intensity or audio level selected to present audible sounds or speech to a patient. An audio intensity may be measured by decibels hearing level (dB HL). The predetermined audio intensity may be based on, for example, the hearing threshold measured for a normal hearing person (e.g., audio intensity of 0-20 dB HL. In one embodiment, the predetermined audio intensity may be 25 dB HL. In another embodiment, the predetermined audio intensity may depend on different parameters, such as the patient's age, and may be set to 25 dB HL for adults (e.g., 12 years or older) and 30 dB HL for children (e.g., between ages 4-6 years). Other values or ranges of values may be selected as a predetermined audio intensity.

The dB HL is a reference value and differs for each sound frequency. This scale corresponds to the average threshold of audibility in adults with normal hearing tested at that same frequency and was developed because the normal hearing person does not hear all tones equally well. The normal hearing threshold range for children may be different from the normal hearing threshold for adults.

For brevity and clarity and without limiting, in the present disclosure a hearing threshold implies the lowest level at which a person can detect a sound 50% of the time at a given frequency. Hearing may be measured by an audiometer that sends tones to each ear through earphones. The patient listens and responds each time a tone is heard. For example, the levels at which the patient can barely hear the tones are the patient's hearing threshold levels. The hearing thresholds, measured in dB, are recorded on a chart referred to as an audiogram, for tones at different pitches or frequencies, measured in Hertz.

The terms cited above denote also inflections and conjugates thereof.

The present disclosure is of an automated hearing test system and related methods. The principles and operation of an automated hearing test system and method according to the present disclosure may be better understood with reference to the drawings and the accompanying description.

Current hearing tests for children are often complex, time-consuming and requiring professional testers, and thus some parents may avoid taking their children to these hearing tests. The automated screening test of the present disclosure may provide hearing surveillance and screening tools which may be adapted for young children, for example between the age of 4-6 years old in order to enable a unified, simple hearing screening test for a large scale community. The present disclosure may provide a hearing screening test that matches the cognitive, motoric and attentional skills expected at the age of the target population. For example, the test task should be interesting and motivating, and the test time duration should not exceed child's attention time limits. An interactive task that includes e.g. pointing at pictures on a touch screen, may be easy and attractive for young children.

Each automated hearing test performed as described in the present disclosure does not require more than one person to be operated, it may be performed by a non-medical person and may be performed in various locations, for example, at a medical facility or a kindergarten.

Test procedures for screening a large-scale community should be uniform across different locations (e.g. states or countries) and independent of a tester's subjective judgment. Using an automated hearing screening method may provide automated presentation of test stimuli, automated scoring, automated determination of test results and automated storing of the test results. It may also enable information management that allows the extraction of statistical data reflecting the characteristics of the entire tested population. This type of data is crucial for quality management as well as for epidemiological research on a regional or a state level.

Simplification of current test procedures requires the reduction of test complexity. Focusing on speech audiometry is advantageous, for example, since speech audiometry reflects everyday hearing function and may test the integrity of the entire hearing system. Furthermore, it may be easier to attract a patient's attention by using words, rather than pure tones.

It is potentially advantageous to determine hearing screening test results using a binary indication (pass/fail). Pass/fail threshold should represent normative hearing range values in any of the pure tone test frequencies. It is potentially advantageous to provide an automated hearing test pass criterion threshold which reflects a combination of the speech reception threshold and speech discrimination norms.

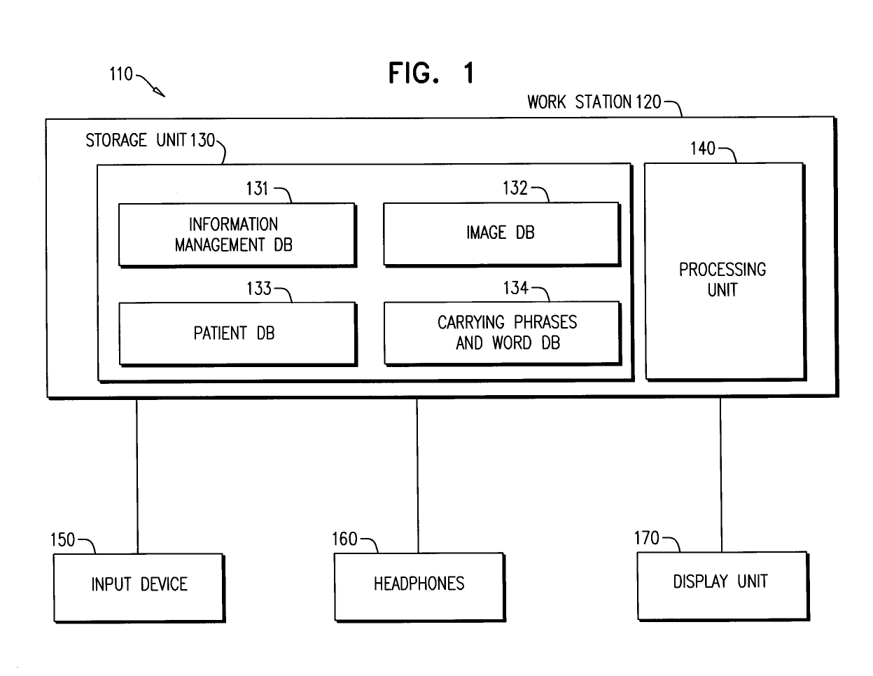

Reference is now made to FIG. 1, which is a schematic illustration of an exemplary embodiment system of for performing an automated hearing test, according to embodiments of the present disclosure. The automated hearing test system 110 may comprise a work station 120, an input device 150, headphones or loudspeaker 160 and optionally an acoustic-to-electric transducer such as a microphone. The work station 120 includes a storage unit 130 and a processing unit 140.

Storage unit 130 is configured to store data which may be used in an automated hearing test. This automatic test is based on displaying images to the patient and introducing corresponding words associated with the images. The test includes detecting a response of a patient and determining whether the response is correct. The number of accumulated correct responses is tracked, and used to determine whether the patient passed or failed the automated hearing test. In case of failing the automated hearing test, an additional hearing test may be necessary.

Storage unit 130 may include (or may be operationally connected to), for example, a patient database 133 which stores information relating to patients. Such information may include a patient's name, identification number, age, address, date of performing one or more automated hearing test, automated hearing test results, and any other data which may be useful to maintain in relation to a patient.

Storage unit 130 may further include, or may be operationally connected to, an information management database 131. The information management database 131 stores information derived from automated hearing test results, and may enable query-based periodical reports, such as number of tests which were performed, the percentage of patients who failed the test, average age being tested and average time for completing a hearing test. Such non-personal data may be reported to governmental or health authorities, e.g. in order to allow quality management and epidemiological research on a regional or state level. Storage unit 130 may further include an image database 132, which comprises a plurality of images which may be displayed to a patient during the hearing test. Any images may be added to the images database 132. The image database 132 may be a folder in a file system, storing the plurality of images, or any other data structure or database as known in the art. The storage unit 130 is adapted to store, e.g. along with each image in the image database 132, one or more corresponding words which are associated to the image.

Storage unit 130 may further include a carrying phrase and word database 134, which stores, for example, carrying phrases which may be audibly presented to a patient. Carrying phrase and word database 134 may store carrying phrases in any desirable language.

Processing unit 140 may be an electronic circuitry that carries out the instructions of a program performing arithmetic, logical, control and input/output (I/O) operations specified by the program instructions. In the present disclosure processing unit 140 is adapted to perform operations as detailed herein.

Processing unit 140 may be configured to select a test-set of images from image database 132. A test-set of images comprises a plurality of images selected from image database 132, each image associated with at least one corresponding word.

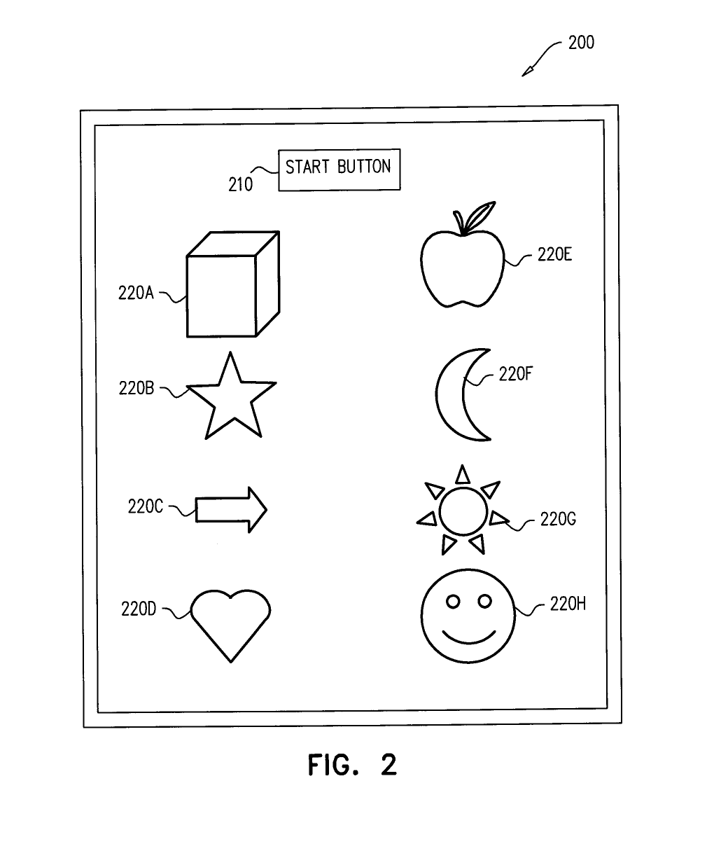

Processing unit 140 may be configured to display the test-set of images to the patient. The selected images may be displayed, for example simultaneously, on a display unit 170 as shown in FIG. 2, in which images 220A-H are displayed.

Processing unit 140 may be configured to select an image from the test-set, for example an image depicting an apple. Processing unit 140 may be configured to cause e.g. headphones 160 or a loudspeaker, to audibly introduce to the patient a carrying phrase. The carrying phrase may be, for example, a carrying phrase randomly selected from the carrying phrase and word database 134. The carrying phrase may be audibly sounded to the patient, e.g. through the headphones 160. Processing unit 140 may be configured to audibly introduce to the patient at least one corresponding word associated with the selected image, for example, for an image depicting an apple, the corresponding word may be “apple”.

Processing unit 140 may be configured to detect an input from the patient; in some cases, processing unit 140 may be configured to detect input during a predetermined time period (timeout period) after audibly presenting the corresponding word. A timeout period may be, for example, within a predetermined range, e.g. between 10 seconds to 30 seconds. A timeout period may be predetermined, or may be set by processing unit 140 according to the number of test iteration that were performed, e.g. a timeout period in a first test iteration may be 5 seconds, a timeout period in a second test iteration may be 10 seconds, etc.

The timeout period may be configurable, and may vary according to various parameters such as the age or the physical abilities of the patient. For example, a patient with a hand disability may need more time to provide an input e.g. by touching a screen or moving a mouse, and as such may require longer timeout periods.

If no input is detected after said timeout period, for example by processing unit 140 and/or by input device 150, processing unit 140 may be configured to select another image from the test-set, and repeat another iteration that includes the operations of audibly introducing the carrying phrase and/or the corresponding word.

After detecting the input from the patient, processing unit 140 may determine whether the patient responded correctly or not. When the image indicated by the patient and provided as input matches the word or words audibly introduced to the patient, the response will be detected as correct. Otherwise, the response will be detected as an incorrect response. For example, if the carrying phrase and corresponding word presented to the patient are “please point at the book”, and the input detected from the patient indicated selecting an image depicting a book from the displayed test-set of images, the response will be determined as a correct response. If the processing unit 140 caused headphone 160 to audibly sound the words “please point at the car” and the input detected from the patient indicated an image depicting a book was selected from the displayed test-set of images, the response will be determined as incorrect.

In the context of the present disclosure, without limiting, a test iteration comprises a set of operations which are performed by a processing unit, e.g. processing unit 140. A test iteration may include one or more of the following operations:
(a) selecting a test-set of images from an image database, the test-set comprising a plurality of images from an image database, each image associated with at least one corresponding word;
(b) displaying the test-set of images to the patient on a display unit;
(c) selecting an image from the test-set;
(d) audibly introducing to the patient a carrying phrase;
(e) audibly introducing to the patient at least one corresponding word associated with the selected image;
(f) detecting input from the patient; and
(g) determining whether the patient responded correctly or not.

The operations of introducing a carrying phrase, and audibly introducing to the patient a corresponding word associated with the selected image, may be generated using the same fixed, predetermined audio intensity. In one embodiment, one or more subsets of operations may be repeated in a test iteration, for example, operations (c)-(g) may be repeated before going back to operation (a).

A test session as referred to herein includes a single automated hearing test which is performed for a single patient. The test session may include a plurality of operations performed by the processing unit 140. A test session may include one or more test iterations which are performed by a single patient. The test session may comprise a predetermined number of successive test iterations performed during a certain time period. The time period defined for a single test session may be for example a predetermined time period or a limited time range, e.g. 4 minutes, or at least 2 minutes, or between 2-5 minutes, etc. A test session may comprise a certain amount of successive test iterations performed by a single patient during a certain time period (referred to as the test session time duration).

In a test session comprising a plurality of test iterations, processing unit 140 calculates the total accumulated number of correct responses that were determined during the test session, and stores (for example in storage unit 130) the accumulated number of correct responses. Processing unit 140 may additionally determine the accumulated number of incorrect responses determined during a test session, and stores the accumulated number of incorrect responses.

If the processor is waiting for input from the patient and no input is detected after completion of a timeout period, processing unit 140 may determine that no response was obtained from the patient. The total number of test iterations during a single test session, for which a patient provided no response, may be calculated (e.g. summed) and further used by processing unit 140.

In one embodiment of the disclosure, one or more test iterations may be performed separately for each ear. For example, a test iteration or test session may be initiated, to determine hearing results of the patient's right ear. In this case, the speech may be sounded using earphones, only to the patient's right ear. A subsequent test session or test iteration may be initiated, to determine the hearing results of the patient's left ear, by sounding the speech (e.g. using earphones) only to the patient's left ear.

In another embodiment, a test session may include alternately testing both ears of the patient, e.g. in a first iteration a first image is selected, and the carrying phrase and corresponding words are audibly introduced only to the right ear, and in the successive iteration in the same test session, the carrying phrase and corresponding words may be audibly introduced to the left ear.

In yet another embodiment, a test session may include alternately testing both ears of the patient, e.g. in a first iteration the carrying phrase may be audibly sounded to both ears, and only the corresponding word/s may be audibly introduced to one ear. In a subsequent iteration, the carrying phrase may be audibly sounded to both ears, and only the corresponding word/s may be audibly introduced to the other ear. This may increase the probability that the patient hears the carrying phrase, even if one ear is weaker and he does not hear the corresponding word which was audibly introduced only to that ear.

Any other order of audibly presenting to a first ear and then to a second ear may be implemented, for example two iterations may be initiated for a first ear, and then two iterations may be initiated for the second ear. In each of the embodiments, the results may be separately calculated for each ear, and the determination whether the patient passed or failed the automated hearing test may be separately provided for the right ear and for the left ear of the patient or may be provided as a single integrated result which indicates the patient's hearing in both ears.

According to some embodiments, the automated hearing test system may comprise also a device for measuring and/or monitoring environmental noise during a test session. In one embodiment the environmental noise may be measured during the test session and may be monitored if it exceeds a predetermined noise threshold. In another embodiment the environmental noise may be taken into consideration, e.g. integrated in the hearing test pass criterion threshold, in order to neutralize or reduce the effect of external noise on the test results.

Processing unit 140 may terminate the test session if the number of successive accumulated incorrect responses exceeds a predetermined incorrect response threshold, or if no input was received from the patient for at least a predetermined number of successive test iterations. The processing unit may generate a related indication to the tester, e.g. that the patient did not understand the task or may be unwilling to cooperate.

Processing unit 140 may determine the accumulated number of correct responses in the performed iterations. When the test session is completed, e.g. if at least a predetermined number of iterations was performed, processing unit 140 may determine whether the patient passed or failed the test session. The result, or an indication to the user or to the tester whether the patient passed or failed the test session, may be presented, e.g. audibly and/or visually displayed, e.g. on the display unit 170.

Reference is now made to FIG. 2, which is a schematic illustration of an exemplary user interface 200 for a hearing screening test, according to embodiments of the present disclosure. By way of example, processing unit 140 may select the following images from image database 132: a box 220A, a star 220B, an arrow 220C, a heart 220D, an apple 220E, a moon 220F, a sun 220G and a face 220H.

Processing unit 140 of FIG. 1 may cause display unit 170 to display the user interface 200, e.g. on display unit 170. The images may be arranged in rows and columns on the screen, e.g. so that each row and each column includes a predetermined number of images. In another embodiment, the images may be arranged circularly, e.g. so that the images are formed as a circle on the displayed screen.

In one embodiment, in each iteration one image is randomly selected from the test-set. The image may be randomly selected only from the group of images of the test-set which were not selected yet in the current test session or may be randomly selected from all images of the test-set. For example, if the test-set includes the following images: a box, a sun, a star and a face, the first image selected randomly may depict a star, the second image may be randomly selected from the group of images including a box, a sun and a face, and excluding the star.

In another embodiment, in each iteration, a new test-set of images may be generated and displayed to the patient. For example, if the test session comprises eight test iterations, eight different test-sets of images will be selected, and in each test iteration a different test-set will be displayed to the patient.

In one embodiment of the disclosure the images are categorized into separate categories within the image database 132, e.g. according to age groups or difficulty levels, and the selection of images to be included in a test-set may be based on the patient's age or cognitive capability. Alternatively, images may not necessarily be divided among different categories, and the selection of the images for the test-set need not be dependent or based on a specific patient's age, cognitive capabilities, or other relevant characteristics.

Before initiating a test session, headphones 160 may be mounted upon and adjusted to the patient's head so that the right earphone 160-r is situated on the right ear and the left earphone 160-1 is situated on the left ear. In addition, a tester may be required to fill out a form including the patient's personal information. An optional embodiment of the disclosure allows choosing a patient's record from a preexisting record set which may be stored, for example, in patient database 133.

The system may be operated by a trained person such as a parent or by a health professional, e.g. a nurse or a doctor, or by an educational professional, e.g. a teacher, who may seat the patient, create a welcoming interaction with the patient and may provide a brief explanation regarding the test session. An appropriate test setting e.g. for children may include a set of children's table and chair in a quiet room designed in a way that assures the child's privacy and prevents external interruptions.

Start button 210 is located in user interface 200 and may be used to initiate a test session, either by the patient or by someone whom the patient is accompanied by, for example, a parent or a nurse.

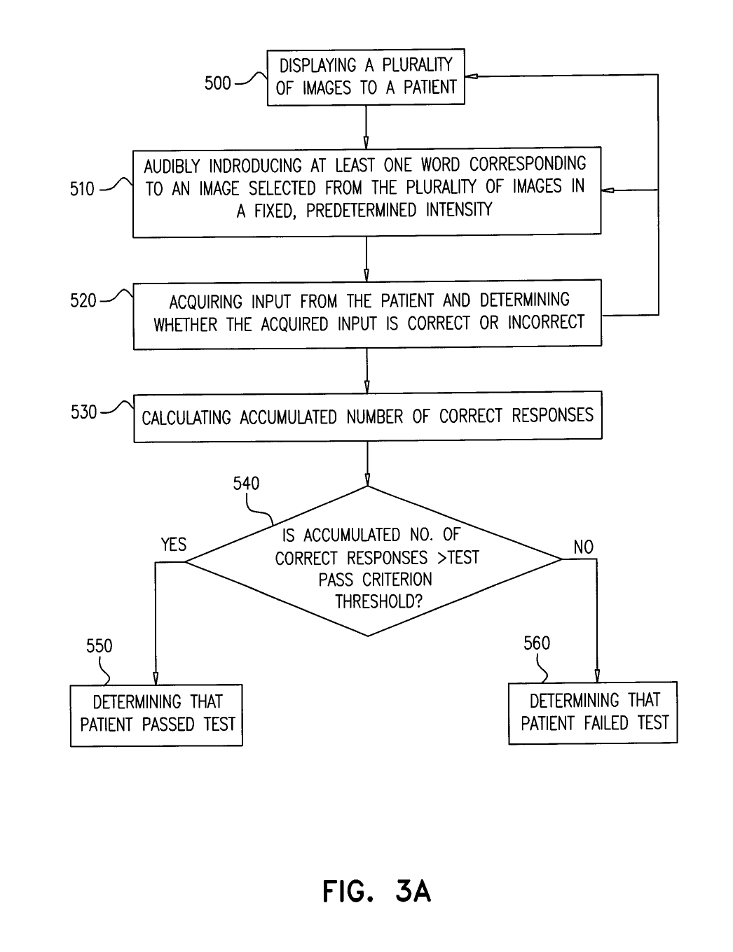

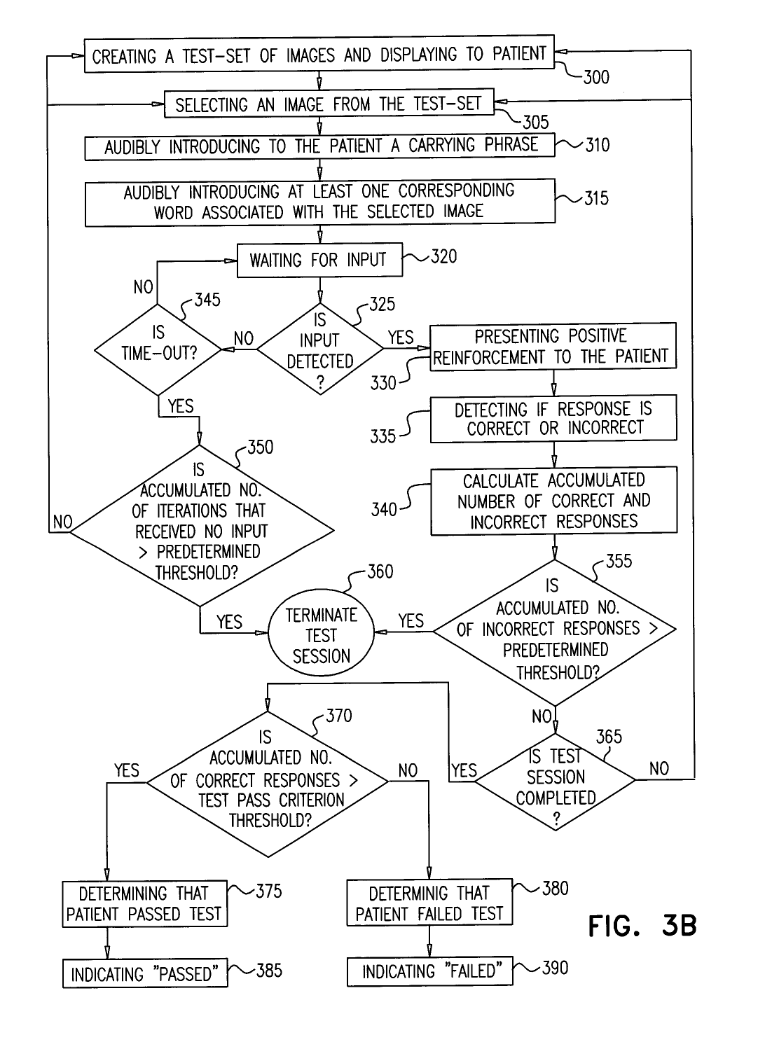

Reference is now made to FIG. 3A which is a flow chart that schematically illustrates performing an automated hearing test method, according to embodiments of the present disclosure.

In operation 500, a set of images may be displayed to a patient, e.g. on a display unit 170. For example, the set of images may include a number of images selected from image database 132 of FIG. 1. In operation 510, after the set of images is displayed, or substantially simultaneously or during the time the set of images is displayed to the patient, at least one word corresponding to an image selected from the plurality of images may be audibly introduced to the patient, e.g. by sounding the corresponding word through headphones 160 of FIG. 1. The corresponding word may be sounded in a fixed, predetermined intensity, for example in 20, 25 28 or 32 dB HL.

In operation 520, input may be acquired from the patient, e.g. through input device 150 of FIG. 1. Processing unit 140 may receive the input, and determine whether the acquired input is correct or incorrect, e.g. whether the patient indicated the image which is related to the corresponding word that was audibly introduced. During a single test session, operations 500-520 may be repeated, for example a predetermined number of times, or at least during a predetermined time duration.

In operation 530, the accumulated number of correct responses may be calculated, e.g. by processing unit 140. In operation 540, the accumulated number of correct responses may be compared to a predetermined threshold, e.g. by processing unit 140. The predetermined threshold may be a test pass criterion threshold, which will be further explained hereinbelow.

If the result of the comparison performed in operation 540 is positive, processing unit 140 may determine that the patient passed the hearing test (operation 550), and may generate a corresponding indication to the patient and/or to the tester, e.g. by displaying a notification on display unit 170 or by audibly sounding a notification. If the result of the comparison performed in operation 540 is negative, processing unit 140 may determine that the patient failed the hearing test (operation 560), and may generate a corresponding indication to the patient and/or to the tester.

Reference is now made to FIG. 3B which is a flow chart that schematically illustrate an automated hearing test method, according to embodiments of the present disclosure.

In operation 300, a test-set of images may be generated or created, for example by selecting a number of images from image database 132 of FIG. 1. (e.g. a predetermined number). The selection of images which are included in a test-set may be a random selection, for example by using known random functions such as Dirichlet process or random permutation. Each of the images in the generated test-set may be simultaneously or substantially simultaneously displayed to a patient on a user interface, e.g. user interface 200 of FIG. 2. Displaying images simultaneously, when referred to herein, may include displaying images during the same time period or substantially the same time period or time duration.

In operation 305, an image may be selected from the displayed test-set of images. The image may be selected randomly, or according to a predetermined order.

In operation 310, a carrying phrase may be audibly introduced to the patient, e.g. by playing the carrying phrase through headphones 160. The carrying phrase may be randomly selected from a collection of carrying phrases (e.g. stored in carrying phrase and word database 134), or may be associated with the selected image. Other methods may be used to select an appropriate carrying phrase. In some embodiments, only one carrying phrase may be used. In other embodiments, corresponding words or images may be associated with carrying phrases, and may be stored accordingly in the carrying phrase and word database 134 or in image database 132. For example, for each image, an indication may be provided in image database 132 regarding one or more carrying phrases which are associated with the image. In some embodiments, a carrying phrase is not required and may not be sounded to the patient.

In operation 315, at least one corresponding word associated with the selected image may be audibly introduced to the patient, for example through headphones 160, using a predetermined audio intensity. Corresponding words are randomly introduced to the patient in order to prevent any spatial clues which may cause the patient to respond correctly even if he/she did not hear the introduced word or sentence. The carrying phrase may also be randomly selected in order to create a natural language flow, and to avoid monotony during the test session.

The corresponding words and carrying phrases may be prerecorded by the same reader, at the same recording settings and presented at the same fixed, predetermined audio intensity level.

An optional embodiment of the disclosure enables to perform a pre-test in order to ensure the selection of words that are familiar to the patient. This option might consume time and attention and is preferred only when testing very young children or children who are not tested in their mother tongue.

In one embodiment of the present disclosure in operations 310 and 315, audibly presenting the carrying phrase and the corresponding word may be performed on one ear only. When test iterations of a first test session are completed, a new test session may be initiated for the second ear. In another embodiment of the present disclosure a test session may comprise test iterations which are performed alternately on both ears, e.g. in the first test iteration the carrying phrase and the corresponding word are audibly introduced to one ear and in the next test iteration the corresponding word are audibly introduced to the second ear. In another example, in the first test iteration the carrying phrase is introduced to both ears, and the corresponding word is audibly introduced to one ear and in the next test iteration the carrying phrase is introduced to both ears, and the corresponding word is audibly introduced to the second ear. This process may be repeated until the test session is complete, or until all images of the test-set were selected, or until any other stopping condition is fulfilled.

As a result of operations 310 and 315 the patient may be audibly presented with a full sentence or a sentence clause in which the verb or verb phrase is the carrying phase, and the subject is a corresponding word associated with a selected image which is being displayed to the patient. The carrying phase along with the corresponding word are meant to prompt the patient to indicate or select one of the plurality of displayed images.

In operation 320, processing unit 140 may wait for an input from the patient. The input may be obtained, for example, using a touch screen, a mouse, a joystick, a keyboard, or any other type of input device. The detected input, accordingly, may be a screen touch indicating an image, a click or a movement of the mouse, etc.

In operation 325, processing unit 140 may determine if an input from a patient has been obtained.

In operation 330, if input from the patient was obtained, processing unit 140 provides a positive reinforcement to the patient, in order to increase the likelihood that the patient will continue to cooperate in the upcoming test iterations or test sessions.

Positive reinforcement includes reinforcing desired behaviors and thereby strengthening a desirable response or behavior. Positive reinforcement may be used in the present disclosure to encourage the patient to cooperate and perform the tasks which comprise the hearing test.

In one embodiment of the present disclosure, when an input of the patient is detected (e.g., either correct or incorrect input), a positive reinforcement is presented to the patient. The positive reinforcement may be audibly sounded, e.g. by using headphones 160, to provide an applause sound or a reinforcing expression, for example, “well done!”. The positive reinforcement may be visually displayed, e.g. by an indication on display unit 170, for example, by activating flashing lights or displaying a smiling face.

In operation 335, processing unit 140 determines whether the obtained input is correct or incorrect. A test iteration response may be scored as a correct response when the patient indicates the correct image, e.g. if the input device is a touch screen, a correct response is determined when the patient touches the area corresponding to the image associated with the corresponding word that was audibly introduced during the test iteration. In another example, when a mouse is used as an input device, a correct response may be determined when the patient clicks a button of the mouse in the display area corresponding to the selected image of the test iteration. In yet another example, the patient may say (e.g. audibly speak) the response, and a speech recognition module (e.g. a machine or program adapted to receive and interpret dictation) may recognize whether the patient's response corresponds to the selected image. If the patient did not respond correctly, the response may be considered an incorrect response.

If no input was detected after a certain period, e.g. a timeout period, the test iteration response may be considered “no response” or an undetermined response.

In operation 340 processing unit 140 calculates an accumulated number of correct responses and incorrect responses that were provided by the patient in all test iterations of the current test session.

Processing unit 140 may determine that no response or an undetermined response was obtained, for example if no input was detected after at least a timeout period (operation 345). In another example, the processing unit 140 may determine that an undetermined response was obtained if the patient provided an undetermined response, e.g. touched or clicked on an irrelevant area or portion of the screen, or said an incoherent word.

The timeout period is a limited time period, for example predetermined or calculated, during which the patient may determine the required response and provide an input using input device 150.

In operation 350 processing unit 140 checks if the accumulated number of iterations that received no input or an undetermined input is larger than an undetermined response threshold. If the accumulated number of iterations that received no input or an undetermined input is larger than the undetermined response threshold, the test session may be terminated. Otherwise, a new iteration may be initiated, e.g. by selecting a new image from the same test set or by generating a new test set of images and displaying it to the patient (e.g., repeating operations 300-365 or 305-365).

In operation 355 processing unit 140 checks if accumulated number of incorrect responses is larger than a threshold, for example a predetermined threshold (e.g., a predetermined incorrect response threshold). If the accumulated number of incorrect responses is larger than a predetermined threshold (e.g., a predetermined incorrect response threshold), the test session is terminated. Otherwise, the processing unit 140 determines if the test session is completed (at operation 365).

In operation 360 the test session is terminated if the accumulated number of incorrect responses is larger than a threshold (e.g., a predetermined incorrect response threshold) or if the accumulated number of iterations to which no input was received is larger than a threshold (e.g., a predetermined undetermined response threshold). For example, if the predetermined incorrect response threshold is four, and four incorrect responses are detected in consecutive test iterations, the test session may be terminated. In other embodiments, a test session may be terminated if the accumulated number of incorrect responses is above the predetermined incorrect response threshold, and the incorrect responses are not necessarily detected in consecutive test iterations. Termination of the test session may include, for example, an indication to the patient and/or to the tester regarding the reason for termination, for example since no input was provided or detected, or since the number of incorrect responses exceeded an allowed or reasonable amount.

In operation 365, processing unit 140 determines whether to repeat operations 300-365, or 305-365, until a stopping condition is fulfilled. In one example, a stopping condition may be if all images of the current test-set were selected and audibly introduced to the patient. In another example, the number of test iterations may be predetermined and not based on the number of images in a test-set.

In operation 370 processing unit 140 may calculate a hearing test result, e.g. by dividing the accumulated number of correct responses by the number of iterations performed during the test session. The result may be displayed as a percentage. For example, a test session may include eight iterations, each iteration including at least selecting one image and audibly introducing the corresponding word to the patient. If four of the patient's responses were correct, the calculated result will be 50%. Further, processing unit 140 may determine whether the result is larger than (or equal to) a test pass criterion threshold.

For brevity and clarity and without limiting, a test pass criterion threshold may be a predetermined result, which is considered sufficiently high to determine that the patient passed the test. Thus, if a patient receives a score lower than the test pass criterion threshold, it may be determined that the patient failed the test. For example, the test pass criterion threshold may be a percentage calculated based on an average number of correct responses that a normal hearing patient should provide in a test session, divided by the number of iterations in a test session.

A test result which is above the test pass criterion threshold indicates that the patient passed the hearing screening test, while a result which is below the test pass criterion threshold indicates that the patient failed the hearing screening test. A test pass criterion threshold may vary according to various parameters. For example, the test pass criterion threshold may vary according to different health regulations that may be applicable for a certain patient group or a community, e.g. a certain age-group of patients. In another example, the test pass criterion threshold may vary according to the sequence of operations performed during the test session, e.g. if the test session is performed for a single ear and not for both ears. In one embodiment, a test pass criterion threshold may be in the range of, for example 70%-90%. In one embodiment the test pass criterion threshold may be preset to 75%.

In operation 375 and 380 processing unit 140 determines if the patient passed (operation 385) or failed (operation 390) the test session. If the calculated result in operation 370 is larger than (or equal to) a test pass criterion threshold processing unit 140 will determine that the patient has passed the test session. Otherwise, processing unit 140 may determine that the patient has failed the test session. For example, if the percentage of correct responses in the embodiment test session is calculated to be 90% and thus larger than the test pass criterion threshold of 75%, the processing unit 140 may determine that the patient passed the automated hearing test. Otherwise, the processing unit 140 may determine that the patient failed the automated hearing test.

In operations 385 and 390 processing unit 140 indicates the test session result, e.g. passed (operation 385) or failed (390) which may be stored in storage unit 130.

In an embodiment in which both ears are tested during a single test session operations 370-390 may be repeated for each ear separately. If only one ear is tested in a test session, two sessions may be performed for a patient, and an indication of which ear has passed or failed may be determined and displayed, e.g. to the patient and/or to the tester.

For each test session, related data such as the results of the test, the test duration, the specific set of words that were introduced to each ear of the patient and patient's input in each iteration, etc. may be stored in information management database 131 or in patient database 133. The stored information may later be used e.g. for further patient monitoring and/or for statistical information analysis.

In some embodiments, the predetermined settings (e.g. thresholds, fixed audio intensity, etc.) may be configurable, and may be set according to different patient characteristics or capabilities. These characteristics may be related to age, cognitive capabilities, physical impairments, mother-tongue, etc.

For example, the number of images in a test-set may be 4, 8 or 16. The selection of the number of images may depend on various factors, e.g. age of the patient, whether the test is performed on both ears alternately in a single test session or test iteration, or for each ear separately in a separate test session or in separate iterations.

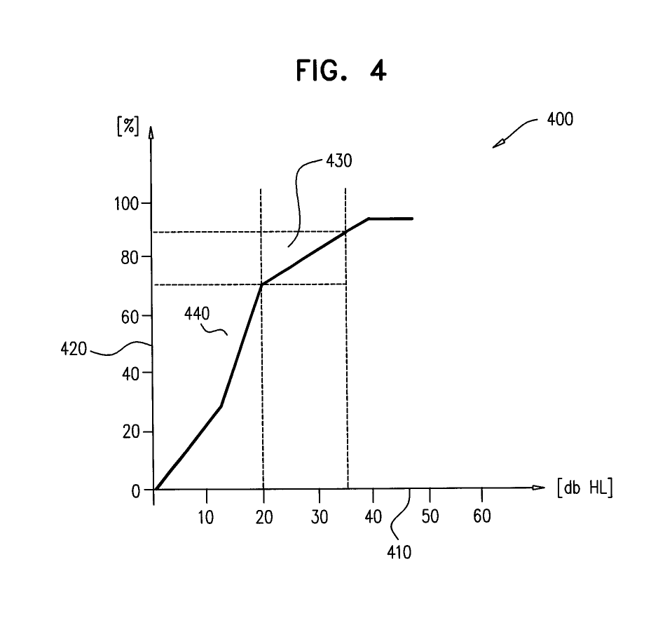

FIG. 4 is a schematic illustration of a performance-intensity function graph, which may be used for determining a test pass criterion threshold for an automated hearing test system according to embodiments of the present disclosure.

For brevity and clarity and without limiting, in the present disclosure a normative performance intensity function indicates the improvement in recognition of spoken words that occurs as the intensity of sound is increased. The performance intensity function plots speech performance in percent of correct responses on the Y-axis, as a function of the level of the speech signal on the X-axis.

Performance intensity function results graph 400 comprises a stimulus intensity axis 410 which is the X axis, and a word recognition score axis 420 which is the Y axis. The word recognition score plot 440 indicates the performance intensity function.

The automated hearing test pass criterion threshold is indicated in a rectangular area 430 which corresponds to an exemplary stimulus intensity level in the range of 20-35 dB HL and a word recognition score in the range of, for example, 70%-90%. Different ranges may be selected, for example, the range of 25-30 dB HL may be selected so that the test tasks are not too easy for a patient to perform. The automated hearing test pass criterion threshold is based on the combination of two norms: a speech reception threshold and a speech discrimination as described hereinafter.

In one embodiment, the test pass criterion threshold may be set to 75%, e.g. if more than one error is input by the patient in a test session comprising 8 iterations, the patient is considered to have failed the test. According to the normative performance intensity function, this score can be expected at an audio intensity level in the range of e.g. 25-30 dB HL. If the fixed audio intensity level is selected to be below 25 dB HL, or in some embodiments below 20 dB HL, the test results may be incorrect or inaccurate. For example, if the audio intensity is 15 dB HL, the expected test results may be in the vicinity of 30% and the patient may be wrongly determined as having a hearing problem.

Setting the test pass criterion threshold to area 430 on the normative performance intensity function may ensure that the patient is able to hear speech at the lowest margins of normal conversation intensity level, and that the patient is able to recognize familiar words spoken to him at such audio intensity.

The automated hearing test offers an alternative to the speech reception threshold testing and the speech discrimination testing in order to provide a pass criterion threshold for testing both hearing sensitivity and hearing accuracy, e.g. in a single hearing test session, performed as described before.

While the disclosure has been described with respect to a limited number of embodiments, it may be appreciated that many variations, modifications and other applications of the disclosure may be made.

The terms ‘processor’ or ‘computer’, or system thereof, are used herein as ordinary context of the art, such as a general purpose processor or a micro-processor, RISC processor, or DSP, possibly comprising additional elements such as memory or communication ports. Optionally or additionally, the terms ‘processor’ or ‘computer’ or derivatives thereof denote an apparatus that is capable of carrying out a provided or an incorporated program and/or is capable to controlling and/or accessing data storage apparatus and/or other apparatus such as input and output ports. The terms ‘processor’ or ‘computer’ denote also a plurality of processors or computers connected, and/or linked and/or otherwise communicating, possibly sharing one or more other resources such as a memory.

The terms ‘software’, ‘program’, ‘software procedure’ or ‘procedure’ or ‘software code’ or ‘software instructions’ or ‘executable code’ or ‘code’ may be used interchangeably according to the context thereof, and denote one or more instructions or directives or circuitry for performing a sequence of operations that generally represent an algorithm and/or other process or method. The program is stored in or on a medium such as RAM, ROM, or disk, or embedded in a circuitry accessible and executable by an apparatus such as a processor or other circuitry.

The processor and program may constitute the same apparatus, at least partially, such as an array of electronic gates, such as FPGA or ASIC, designed to perform a programmed sequence of operations, optionally comprising or linked with a processor or other circuitry.

The term computerized apparatus or a similar one denotes an apparatus having one or more processors operable or operating according to a program.

As used herein, without limiting, a module represents a part of a system such as a part program operating together with other parts on the same unit, or a program component operating on different unit, and a process represents a collection of operations for achieving a certain outcome.

The term “configuring” and/or ‘adapting’ for an objective, or a variation thereof, implies using at least a software and/or electronic circuit and/or auxiliary apparatus designed and/or implemented and/or operable or operative to achieve the objective.

A device storing and/or comprising a program and/or data constitutes an article of manufacture. Unless otherwise specified, the program and/or data are stored in or on a non-transitory medium.

In case electrical or electronic equipment is disclosed it is assumed that an appropriate power supply is used for the operation thereof.

The flowchart and block diagrams illustrate architecture, functionality or an operation of possible implementations of systems, methods and computer program products according to various embodiments of the present disclosed subject matter. In this regard, each block in the flowchart or block diagrams may represent a module, segment, or portion of program code, which comprises one or more executable instructions for implementing the specified logical function(s). It should also be noted that, in some alternative implementations, illustrated operations may occur in deferent order or as concurrent operations instead of sequential operations to achieve the same or equivalent effect.

The corresponding structures, materials, acts, and equivalents of all means or step plus function elements in the claims below are intended to include any structure, material, or act for performing the function in combination with other claimed elements as specifically claimed. As used herein, the singular forms “a”, “an” and “the” are intended to include the plural forms as well, unless the context clearly indicates otherwise. It will be further understood that the terms “comprises” and/or “comprising” and/or “having” when used in this specification, specify the presence of stated features, integers, steps, operations, elements, and/or components, but do not preclude the presence or addition of one or more other features, integers, steps, operations, elements, components, and/or groups thereof.

As used herein the term “configuring” and/or ‘adapting’ for an objective, or a variation thereof, implies using materials and/or components in a manner designed for and/or implemented and/or operable or operative to achieve the objective.

The terminology used herein should not be understood as limiting, unless otherwise specified, and is for the purpose of describing particular embodiments only and is not intended to be limiting of the disclosed subject matter. While certain embodiments of the disclosed subject matter have been illustrated and described, it will be clear that the disclosure is not limited to the embodiments described herein. Numerous modifications, changes, variations, substitutions and equivalents are not precluded.

What is claimed is:

1. A computer-implemented method for performing a hearing test of a patient, comprising:
repetitively performing a plurality of iterations, each iteration comprising:
displaying a plurality of images and consequently audibly introducing to the patient by sounding in a fixed predetermined audio intensity at least one word corresponding to at least one image of the plurality of images, and
responsive to the sounding the at least one word, acquiring from the patient at least one input, said input indicative of the at least one image;
and
determining a result of the hearing test based on a number of correct inputs provided by the patient.

2. The method according to claim 1, comprising selecting a test-set of images from an image database, the test-set comprising a plurality of images from an image database, each image associated with an at least one word corresponding to the image.

3. The method according to claim 2, wherein the images of the test-set are displayed to the patient on a display unit.

4. The method according to claim 3, wherein each iteration further comprises selecting the at least one image from the test-set.

5. The method according to claim 1, wherein each iteration further comprises audibly introducing to the patient a carrying phrase using the fixed predetermined audio intensity.

6. The method according to claim 1, comprising detecting whether input is acquired from the patient.

7. The method according to claim 6, comprising determining whether the acquired input is correct or incorrect.

8. The method according to claim 1, comprising repeating each iteration a predetermined number of times.

9. The method according to claim 4, wherein the selected image from the test-set is randomly selected.

10. The method according to claim 1, wherein the plurality of images is displayed while the carrying phrase and the at least one word corresponding an image are audibly introduced to the patient.

11. The method according to claim 1, wherein the predetermined intensity is calculated based on normal hearing of the ranges of a population to which patient belongs.

12. The method according to claim 11, wherein the predetermined intensity is in the range of 20-35 DB HL.

13. The method according to claim 11, wherein the predetermined intensity is in the range of 25-30 DB HL.

14. The method according to claim 1, further comprising detecting ambient noise in the test environment.

15. The method according to claim 14, wherein determining a result of the hearing test is also based on the detected ambient noise.

16. A computerized system for a hearing test of a patient, comprising:
an audio sounding device; and
a processing unit configured to repetitively perform a plurality of iterations,
wherein in each iteration the processing unit is configured to:
display a plurality of images and consequently audibly introduce to the patient by sounding in a fixed predetermined audio intensity at least one word corresponding to at least one image of the plurality of images, and
responsive to the sounding the at least one word, acquiring from the patient at least one input, said input indicative of the at least one image;
and
the processing unit further configured to determine a result of the hearing test based on a number of correct inputs provided by the patient.

17. The system according to claim 16, further comprising an input device for providing the input indicative of the at least one image by the patient.

18. The system according to claim 16, wherein the input device is selected from a touch screen, a keyboard, a joystick and a mouse.

19. The system according to claim 16, wherein the processing unit is configured to randomly select the image from the test-set.

20. The system according to claim 19, wherein the processing unit is configured to terminate the hearing test if a certain predetermined number of successive incorrect responses are detected, or if no response is detected in at least a predetermined number of iterations.
